(19) United States
(12) Patent Application Publication
(10) Pub. No.: US 2010/0086485 A1
(43) Pub. Date: Apr. 8, 2010

(54) INFLUENZA VACCINE

(76) Inventor: Heiko Apfel, Pulheim (DE)

Correspondence Address: ROTHWELL, FIGG, ERNST & MANBECK, P.C., 1425 K STREET, N.W., SUITE 800, WASHINGTON, DC, 20005, US

(21) Appl. No.: 12/161,261

(22) PCT Filed: Jan. 17, 2007
(86) PCT No.: PCT/EP07/00386

§ 371 Date: Jul. 17, 2008

(30) Foreign Application Priority Data
Jan. 17, 2006 (EP) 06 000 951.1

(51) Int. Cl. A61K 49/00; C12N 1/21; C12N 15/63; A61K 35/74; C07K 14/00; A61K 39/145; C40B 40/02; A61K 31/7052; A61P 31/16

(52) U.S. Cl. 424/9.2; 435/252.3; 435/320.1; 424/93.2; 530/350; 424/206.1; 506/14; 514/44 R

(57) ABSTRACT

The present invention relates to a live-attenuated bacterial cell comprising a heterologous nucleotide sequence encoding at least one influenza virus antigen in operative linkage to an expression system.

The present invention relates to a live-attenuated bacterial cell comprising a heterologous nucleotide sequence encoding at least one influenza virus antigen in operative linkage to an expression system.

Influenza is caused by a single stranded RNA virus of the family Orthomyxoviridae. The virus is divided into the three main types A, B, C which are distinguished by differences in two major internal proteins, nucleoprotein (NP) and matrix protein 1 (MP1). Influenza virus type A is the most significant epidemiologically, because it is found in a wide variety of bird and mammal species and undergo major shifts in immunological properties. Influenza A is further divided into subtypes based on differences in the membrane proteins hemagglutinin (HA) and neuraminidase (NA), which are the major targets for the immune system. In total 15 HA subtypes (H) and 9 NA subtypes (N) are known. Major human subtypes are H1N1, H1N2, H2N2 and H3N2. Wild birds contain all subtypes and it is believed that wild birds are a natural reservoir for new virus variants that are transmitted to domestic birds and mammals. It has been further proposed that pigs might be important in facilitating the generation of new virulent variants for humans, since it is known that both avian and human viruses grow well in pigs. Recently, it has been established that avian flu viruses (H5N1) can even infect humans without passage through an intermediate host and without acquiring gene segments from human flu viruses. Influenza epidemics are associated with amino acid changes in the antigenic sites of the major surface antigens HA and NA (antigenic drift). Major pandemics have resulted from introduction of HA and/or NA genes from an animal derived influenza virus, by reassortment, into the genetic background of a currently circulating virus (antigenic shift).

Avian influenza (AI) is a viral disease of fowl that causes a wide range of disease signs. There are two main AI patho-types: highly pathogenic AI (HPAI) and low pathogenic AI (LPAI) virus infections. HPAI infections which are caused by some viruses of the H5 and H7 subtype, usually result in multiorgan systemic disease, with high rates of morbidity and mortality. It has been proved that highly pathogenic avian influenza virus emerge in domestic poultry from LPAI progenitors of H5 and H7 subtype. LPAI viruses are associated with mild respiratory disease and reduction in egg production (Alexander, 2000; Swayne, 2003). After infection, the virus is excreted from both the respiratory and the digestive tracts, resulting in the rapid spread through a population of susceptible hosts. Wild water fowl and shorebirds provide a reservoir for all 15 influenza A virus hemagglutinin (HA) subtypes. Infection in wild birds is generally asymptomatic and mainly restricted to the intestine.

The appearance of novel variants from natural reservoirs remains a major threat for humans and livestock. Accordingly, there is a strong need to protect poultry flocks from AI virus infections transmitted by wild fowls.

Avian influenza virus subtypes have high prevalence worldwide and some variants even have the potential to infect human beings by direct infection or by deriving from an intermediate host which is able to propagate both avian and human influenza virus, like pigs. The development of a hetero-subtypic AI virus vaccine is therefore highly recommended for all kind of domestic birds in order to prevent transmission of AI virus to humans.

Presently, there is no vaccine available which mediates broad-spectrum protection against the most relevant AI virus subtypes. Whenever an AI virus epidemic appears trading bans are declared by the governments and the infected animals are stamped-out. If an appropriate vaccine is available, the uninfected flocks are prophylactically vaccinated in order to suppress virus circulation. The proof that an infection has stopped circulation in the vaccinated population is a prerequisite to lift trade bans.

The general problem of influenza virus vaccine development is the high antigenic variance which originates from (1) the inaccuracy of the viral replication system which generates amino acid changes in the prominent antigenic sites (drift mutants) and (2) the fragmentation of the genome which becomes re-assorted in mixed infections (shift mutants). The conventional anti-AI virus vaccines are prepared from a homo-subtypic field isolate which are either used as inactivated whole virus vaccine or as fractionated (split) vaccine. If a new virus subtype appears the production of a novel homo-subtypic vaccine strain is demanded which takes some time. Almost every year a novel subtypic virus variant appears which renders the conventional vaccination strategy almost inefficient. The administration of a mixture of different vaccine subtypes might be an alternative. However, the use of whole virus vaccines on the basis of homo-subtypic field isolates provokes immune responses which are almost identical to natural infections. This hampers the discrimination of vaccinated animals from infected animals which impedes the use of such vaccines. Re-assortant vaccines have been prepared which share with the circulating virus either one or two of the genomic segments or only one defined genomic segment of unrelated subtype, e.g. encoding NA. This helps to differentiate infected animals from vaccinated animals (DIVA) by serological tests. However, the manufacturing of such vaccines still takes much time which impedes rapid vaccination measures if a novel virus variant appears. Unless it is possible to predict the relevant subtype and the matching vaccine is produced in advance.

The major target antigen of protective immunity against influenza virus infection, HA, is hyper-variable. Nevertheless, partial protection has been observed after repeated immunization using different subtypes of influenza. It has been further demonstrated that escalating doses of HA correlate with increasing cross-reactivity of anti-HA immunity which improves neutralisation of subtypic (drift) virus variants. This effect is improved when the major surface antigen HA is combined with the second most surface antigen, NA, which has a slower rate of antigenic variation. Anti-influenza immune responses mediated by cytotoxic lymphocytes (CTL) are known to reduce the intensity and duration of viral shedding. For the induction of CTL immune responses invariant viral antigens seems to be of relevance. These antigens are localised inside the viral particle or beneath the HA-NA surface layer and become accessible for the immune system during virus replication in epithelial cells. The protective potential of these conserved antigens has been demonstrated in various experimental models using nucleoprotein (NP), matrix protein 1 and 2 (M1, M2) and combinations thereof with other viral antigens. None of these approaches are in use but they have been approved to be at least partially protective under controlled laboratory conditions.

Invading virus particles are most efficiently defeated by secretory IgA which bind to the virus surface and subsequently cause agglutination of virus particles. The major target antigen is HA which appears on the virus surface as homopolymeric structure. This implies rather complex antibody binding motifs involving several HA-subunits. In order to stimulate effectual neutralising antibodies, the vaccine must contain almost native antigens. For this reason inactivated whole virus vaccines or live-attenuated influenza virus vaccines (e.g. a cold-adapted strain) are commonly used. Novel vaccine approaches use recombinant viral or plasmid vectors which encode the viral antigens of interest. The delivered vectors actively (viral vector) or passively (plasmid vector) enter cells in the tissue of the vaccinated host where the encoded viral genes are expressed in an almost native configuration and subsequently presented to the host immune system.

Influenza virus is generally transmitted via air and by droplets which are deposited in the mucosa of the respiratory (human and birds) and digestive tract (birds). The first line of specific immune response comprises sIgA and dIgA which are produced in the mucosa and which actually prevent the virus to enter the host. If the virus prevails and continues infection, IgG is responsible for virus neutralisation whereas CTLs eliminate infected cells and release cytokines which control virus infection. Ideally, an anti-influenza vaccine provokes strong mucosal (sIgA/dIgA) and systemic (IgG and cellular) immune responses. Both conventional anti-influenza vaccines and even novel approaches are mostly administered parenterally which provoke excellent systemic immune responses but only minor mucosal immune response.

In order to efficiently provoke mucosal immune system, the vaccine must be delivered either nasally or orally. The favourite mucosal vaccination procedure for animals kept in mass stocks is the oral route. Oral vaccines can be easily administered by drinking water whereas nasal vaccination of small animals kept in mass stocks requires special devices. The most effectual oral vaccines in poultry are live vaccines. Fowlpox Virus, ILT virus (Infectious Laryngotracheitis virus) and live-attenuated Salmonella have been used as a carrier for the oral delivery of foreign antigens. Mostly preferred are viral vectors which yield AI virus antigens that are almost identical to original virus proteins guaranteeing efficient immune response to virus particles. In contrast to this, Salmonella are unable to imitate the post-translationally processes which happens in a eukaryotic cell. Accordingly, Salmonella does not deliver originally shaped AI virus antigens which consider Salmonella inappropriate for conventional anti-influenza vaccination strategies.

The rate for spontaneous mutation among Influenza virus is one mutation per genome per replication cycle or alternatively 1.5×10⁻⁵ mutations per nucleotide per infection cycle. This rate is close to the maximum value compatible with viability (Eigen, 1977). Influenza viruses are using this high genetic variance to escape protective immune response, e.g. neutralisation by antibodies. Such escape mutants appear under laboratory conditions at frequencies of 10⁻⁴ to 10⁻⁴ when virus replicates in presence of a neutralising monoclonal antibody against HA (Nakajima, S. (1981); Webster, R. (1980); Yewdell, J. (1986)). This antigenic variance of HA is confirmed by the analysis of postinfection human sera which revealed polyclonal antibodies with various specificities toward the HA antigen (Nakajima, S. (2000); Wang, M.-L. (1986)). On the basis of these data one may assume that the use of whole protein antigens in vaccination against influenza virus even favours the generation of new virus variants.

Nevertheless, the variance in the primary structure of a protein is limited by the structural requirements which make up the conserved physico-chemical characteristics that are needed to maintain the function of a protein (Afonnikov, D. A. (2001)). Accordingly, an alternative anti-influenza vaccination strategy might consider segments of influenza proteins which are essential for protein function and virus viability. The presently known amino acid substitutions of influenza antigens are representing changes which are not affecting the function of the HA protein and virus viability. On the other hand, there is almost no information whether the amino acids which remained unchanged in HA and other-viral proteins are really essential for protein function. Recently, some evidence was provided that even protein segments of HA which maintain functional capability (essential protein domains) have variable primary structure (Nakajima, K. (2005)). In order to identify essential protein domains of influenza virus antigens with protective potential against influenza infection one might expect much experimental effort.

The present invention describes an approach for the immunological analysis of potentially essential protein domains of various influenza target antigens in order to identify effectual combinations of essential protein domains which are able to mediate protection against hetero-subtypic influenza infection. The potentially essential protein domains of a target antigen are predicted within a group of influenza viruses, preferably influenza A viruses, e.g. within the group of AI viruses including HPAI viruses, such as H5N1, H5N2, H7N3, H7N7, H10N4, and H10N5 and virulent LPAI viruses. After the identification of potentially essential protein domains of a target antigen, the encoding DNA sequences of these domains may be combined, resulting in a chimeric protein which is called a polytope. The polytope is expressed in a bacterial cell, in particular a gram negative bacterial cell, more particularly a Salmonella enterica vaccine strain which is used to evaluate its immunogenic properties, in particular the protection efficacy in specific animals by vaccination experiments. This process may be conducted with various influenza target proteins. In a final step, the most effectual polytopes from different influenza target proteins may be combined and tested for their efficacy to protect animals against influenza infection. The identified amino acid sequences comprising the protective protein domains may be used to manufacture vaccines against influenza virus infections, particularly against infections caused by AI viruses, most particularly against infections caused by HPAI viruses.

The various polytopes are preferably expressed in a plasmid-based system which may be optimised for routine testing of numerous antigens. The plasmid expression system is preferably conceived for Salmonella enterica ssp. vaccine strains.

The bacteria of the present invention preferably express the polytopes of a given influenza antigen or combined polytopes from different influenza proteins and preferably expose these polytopes onto the bacterial surface.

Orally delivered Salmonella live vaccine carriers are able to stimulate simultaneously both mucosal (sIgA), humoral and cellular immune responses. It has been reported that orally delivered Salmonella live vaccine carriers are able to stimulate the distant nasal associated lymphatic tissue (NALT) which may render Salmonella as an ideal immune stimulant to control influenza virus replication. Particularly, the Salmonella approach seems to be suitable for defeating AI virus infection which starts in the mucosa of the upper and lower respiratory tract and in the intestinal mucosa of fowls.

In contrast to other oral antigen delivery systems the live attenuated bacterial cell approach of the present invention, in particular the Salmonella approach is simple to manufacture which allows the rapid immunological analysis of numerous antigens. Moreover the approach is applicable in different hosts, e.g. mice, poultry, pigs and even humans, by simply transferring the expression plasmid into an appropriate bacterial vaccine strain, in particular a Salmonella vaccine strain. Thus the protective effect of an antigen or an antigen combination identified in a specific host-virus complex, e.g. fowls experimentally infected with various AI virus subtypes, can be further tested in pigs which are experimentally infected with various subtypes of AI virus or porcine influenza viruses or even influenza viruses originating from humans. For basic immunological analysis in mice the expression plasmid can be propagated in Salmonella enterica vaccine strain of serovar Typhimurium. For protection experiments in fowls the expression plasmid can be transferred into a vaccine strain deriving from Salmonella enterica enterica serovar Gallinarum or Pullorum, e.g. S. Gallinarum 9R. Suitable Salmonella vaccine strains for vaccination experiments in pigs are selected from the serovar Choleraesius. For humans the Salmonella enterica enterica serovar Typhi is recommended, e.g. S. typhi Ty21a.

Another aspect of the present invention is a method for identifying novel polytopic antigens for a heterosubtypic vaccine, in particular for a heterosubtypic anti AI virus vaccine, or/and for an immunogenic composition, in particular for poultry, comprising the steps:
(1) identification of potentially essential amino acid sequences of an influenza virus, in particular an AI virus;
(2) providing a polytope comprising at least two amino acid sequences of (1);
(3) determination if the polytope of (2) is capable of provoking an immune response, in particular a specific immune response, against the original viral protein or/and the influenza virus from which the at least two amino acid sequences of (2) are derived;
(4) optionally evaluating which of the immuno stimulatory polytopic antigens of (3) are capable to suppress or protect vaccinated birds or/and non-human mammals against influenza virus challenge infection, in particular AI virus challenge infection:

In the method for identifying novel polytopic antigens of the present invention, the potentially essential amino acid sequences may be selected from any influenza virus polypeptide, e.g. from hemagglutinin, neuraminidase, matrix protein M1, matrix protein M2, non-structural protein 1, non-structural protein 2, nucleoprotein, and polymerase complex proteins. Preferably, the potentially essential amino acid sequences are selected from the hemagglutinin (HA) or/and the matrix protein 2 (MP2). Both HA and MP2 appear on the surface of infected cells and become accessible for the host immune system.

Potentially essential amino acid sequences can also be selected from the N-terminus of the HA2 subunit (HA2ₐₐ₁₋ₐₐ₂₄). An example of a sequence derived from HA2ₐₐ₁₋ₐₐ₂₄ is SEQ ID NO:1.

| | | |
|---|---|---|
| [HA2_{FP1}] | G-L-F-G-A-I-A-G-F-I-E | (SEQ ID NO: 1) |

Potentially essential amino acid sequences can also be selected from α-helical clusters, in particular from the sequence HA2ₐₐ₃₈₋ₐₐ₁₂₉, more particular from the sequence HA2ₐₐ₄₀₋ₐₐ₁₀₅. Examples of potentially essential amino acid sequences selected from HA2ₐₐ₄₀₋ₐₐ₁₀₅ are SEQ ID NO:2, SEQ ID NO:3, SEQ ID NO:4, and SEQ ID NO:5.

| | | |
|---|---|---|
| [HA2_{Helix1a}] | K-A-I-D-K-I-T-S-K | (SEQ ID NO: 2) |
| [HA2_{Helix1b}] | K-A-I-D-G-I-T-N-K | (SEQ ID NO: 3) |
| [HA2_{Helix1c}] | K-A-I-D-G-V-T-N-K | (SEQ ID NO: 4) |
| [HA2_{Helix1d}] | S-A-I-D-Q-V-T-G-K | (SEQ ID NO: 5) |

Potentially essential amino acid sequences can also be selected from the ectodomain (eMP2) of MP2. Examples of potentially essential amino acid sequences selected from the eMP2 region are

| | |
|---|---|
| [eMP2₁] | |
| M-S-L-L-T-E-V-E-T-L-T-R-N-G-W-G | (SEQ ID NO: 6) |
| [eMP2₂] | |
| M-S-L-L-T-E-V-E-T-P-T-R-N-E-W-E | (SEQ ID NO: 7) |

A preferred essential amino acid sequence is selected from SEQ ID NO:1, SEQ ID NO:2, SEQ ID NO:3, SEQ ID NO:4, SEQ ID NO:5, SEQ ID NO:6, and SEQ ID NO:7.

Yet another aspect of the present invention is an epitope.

The epitope may be selected from the potentially essential amino acid sequences described herein. The epitope is preferably selected from HA2ₐₐ₁₋ₐₐ₂₄, HA2ₐₐ₃₈₋ₐₐ₁₂₉, HA2ₐₐ₄₀₋ₐₐ₁₀₅, and the ectodomain of MP2. More preferably, the epitope is selected from SEQ ID NO:1, SEQ ID NO:2, SEQ ID NO:3, SEQ ID NO:4, SEQ ID NO:5, SEQ ID NO:6, and SEQ ID NO:7.

The epitope may have a length of at least 5 up to 50 amino acid residues, preferably at least 5 up to 20 amino acid residues or of a at least 10 up to 30 amino acid residues.

Yet another aspect of the present invention is a polytopic antigen (herein also referred to as polytope). In the present invention, a polytopic antigen or polytope may comprise a plurality of epitopes, e.g. 2 to 50 epitopes, such as 2 to 10 epitopes, 5 to 20 epitopes, or 10 to 40 epitopes.

The epitopes in a polytopic antigen or polytope may independently be selected from the potentially essential amino acid sequences described herein. The epitopes in a polytopic antigen or polytope preferably comprise sequences independently selected from HA2ₐₐ₁₋ₐₐ₂₄, HA2ₐₐ₃₈₋ₐₐ₁₂₉, HA2ₐₐ₄₀₋ₐₐ₁₀₅, and the ectodomain of MP2. More preferably, the epitopes are independently selected from SEQ ID NO:1, SEQ ID NO:2, SEQ ID NO:3, SEQ ID NO:4, SEQ ID NO:5, SEQ ID NO:6, and SEQ ID NO:7.

A polytopic antigen or polytope of the present invention may comprise any combination of SEQ ID NO:1, SEQ ID NO:2, SEQ ID NO:3, SEQ ID NO:4, SEQ ID NO:5, SEQ ID NO:6, and SEQ ID NO:7.

The polytopic antigen or polytope of the present invention may comprise at least two, at least three, at least four, at least five, or at least six sequences selected from SEQ ID NO:1, SEQ ID NO:2, SEQ ID NO:3, SEQ ID NO:4, SEQ ID NO:5, SEQ ID NO:6, and SEQ ID NO:7.

The polytopic antigen or polytope of the present invention may also independently comprise at least two copies, at least three copies, at least four copies, at least five copies, at least 10 copies, at least 20 copies, or even more copies of an epitope selected from SEQ ID NO:1, SEQ ID NO:2, SEQ ID NO:3, SEQ ID NO:4, SEQ ID NO:5, SEQ ID NO:6, and SEQ ID NO:7.

The polytopic antigen or polytope of the present invention may also independently comprise spacer sequences (SS) inserted between adjacent or proximate epitopes in order to avoid the generation of extra-epitopes which may result from amino acid sequences present in different adjacent epitopes. The polytopic antigen or polytope of the present invention may comprise at least one, at least three, at least four, at least five, at least six or even more spacer sequences. The spacer sequence may have a length of 1 to 20, 2 to 15, 3 to 10, 4 to 8, or 5 to 7 amino acids. A preferred spacer sequence comprises the amino acid sequence G-P-G-P-G (SEQ ID NO: 9).

The epitope or/and polytopic antigen or polytope of the present invention may also comprise a stimulatory polypeptide (IS) in order to further improve specific immune response in particular in an avian host. A preferred immuno-stimulatory polypeptide comprises the serial amino acid sequence R-K-K-R-R-Q-R-R-R-R-A-A-A (SEQ ID NO: 10) which represents a protein transduction domain. The immuno-stimulatory polypeptide (IS) is preferably located at the N-terminus of the polytopic antigen or polytope of the present invention.

Preferred polytopic antigens or polytopes of the present invention comprise at least one of the structures:

| | |
|---|---|
| Polytope 1 | [eMP2₁]:[eMP2₂]:[HA2_{FP1}] |
| Polytope 1.1 | [eMP2₁]:[SS]:[eMP2₂]:[SS]:[HA2_{FP1}] |
| Polytope 1.2 | [IS]:[eMP2₁]:[SS]:[eMP2₂]:[SS]:[HA2_{FP1}] |
| Polytope 1.3 | [IS]:[eMP2₁]:[eMP2₂]:[HA2_{FP1}] |
| Polytope 2 | [eMP2₁]:[eMP2₁]:[eMP2₂]:[eMP2₂]:[HA2_{FP1}]:[HA2_{FP1}] |
| Polytope 2.1 | [eMP2₁]:[SS]:[eMP2₁]:[SS]:[eMP2₂]:[SS]:[eMP2₂]:[SS]: |
| | [HA2_{FP1}]:[SS]:[HA2_{FP1}] |
| Polytope 2.2 | [IS]:[eMP2₁]:[SS]:[eMP2₁]:[SS]:[eMP2₂]:[SS]:[eMP2₂]: |
| | [SS]:[HA2_{FP1}]:[SS]: [HA2_{FP1}] |
| Polytope 2.3 | [IS]:[eMP2₁]:[eMP2₁]:[eMP2₂]:[eMP2₂]:[HA2_{FP1}]: |
| | [HA2_{FP1}] |
| Polytope 3 | [HA2_{Helix1a}]:[HA2_{Helix1b}]:[HA2_{Helix1c}]:[HA2_{Helix1d}] |
| Polytope 3.1 | [HA2_{Helix1a}]:[SS]:[HA2_{Helix1b}]:[SS]:[HA2_{Helix1c}]:[SS]: |
| | [HA2_{Helix1d}] |
| Polytope 3.2 | [IS]:[HA2_{Helix1a}]:[SS]:[HA2_{Helix1b}]:[SS]:[HA2_{Helix1c}]: |
| | [SS]:[HA2_{Helix1d}] |
| Polytope 3.3 | [IS]:[HA2_{Helix1a}]:[HA2_{Helix1b}]:[HA2_{Helix1c}]:[HA2_{Helix1d}] |

A preferred polytope or polytope combination comprises polytopes 1, 1.1, 1.2, or/and 1.3.

The epitope or/and polytopic antigen or polytope of the present invention may be used for the manufacture of a medicament for the prevention, treatment or/and alleviation of an influenza virus infection, preferably of an influenza A virus infection, more preferably of an influenza A virus infection selected from human influenza A virus subtypes and AI viruses, even more particularly selected from LPAI or HPAI. The influenza virus infection may be an infection with a virus selected from human or avian influenza A virus subtypes H1N1, H1N2, H2N2, H3N2, and avian influenza A virus subtypes H5N1, H5N2, H7N3, H7N7, H10N4 and H10N5.

The bacterial cell as described herein may comprise epitope or/and a polytopic antigen or polytope as described herein or/and a sequence encoding an epitope or/and a polytopic antigen or polytope as described herein.

The sequence encoding an epitope or/and a polytopic antigen or polytope is in operative linkage to an expression system. Preferably, the expression system comprises at least one vector. The vector may be an extrachromosomal vector, e.g. a plasmid or a chromosomal vector, e.g. a vector which integrates into the chromosome of a bacterial host cell. Suitable bacterial vectors are e.g. described in Sambrook et al., Molecular Cloning, A Laboratory Manual. The vector as described herein may comprise a sequence encoding an epitope or/and a polytopic antigen or polytope as described herein.

Further, the invention refers to a pharmaceutical composition, preferably a vaccine composition, more preferably a heterosubtypic vaccine.

A preferred pharmaceutical composition of the present invention is a vaccine composition for the prevention or/and treatment of influenza in humans, in particular a heterosubtypic vaccine for the prevention or/and treatment of human influenza.

Another preferred pharmaceutical composition of the present invention is a vaccine composition for the prevention or/and treatment of avian influenza (AI), in particular a heterosubtypic vaccine for the prevention or/and treatment of avian influenza.

A heterosubtypic vaccine is a vaccine which e.g. exhibits cross-protectivity among different subtypes of influenza, for instance avian influenza.

The pharmaceutical composition may e.g. comprise a live-attenuated bacterial cell as described above or an isolated polytopic antigen or polytope as described above or a combination thereof.

The bacterial cell or the epitope or/and polytopic antigen or polytope of the present invention may be used for the manufacture of a medicament for the prevention or/and treatment of an influenza virus infection, in particular an avian influenza (AI), which medicament may be a vaccine composition or/and an immunogenic composition, in particular a heterosubtypic vaccine composition.

The medicament may be manufactured for prevention, treatment or/and alleviation of virus A influenza in human, companion animals or/and life stock such as pig or/and fowls. A medicament for humans is preferred. Another preferred medicament is for use in fowls.

Another aspect of the present invention is an expression system capable of expressing the epitope or/and polytope or polytopic antigen as described herein in a bacterial cell as described herein.

The expression system of the present invention can be used for optimization of expression of the prepared epitopes or/and polytopes in the various Salmonella vaccine strains approved for poultry.

The expression system of the present invention preferably comprises at least one genetic module selected from transcription module (P_{M}), translation module (SD), translocation module (LP), antigenic module (AIV-Ag), carrier module (LP:OMD)). It is more preferred that the expression system comprises all of these genetic modules, which are in particular suitable for adjustment of the expression and translocation of the epitopes or/and polytopes onto the surface of the individual bacterial strains, in particular Salmonella vaccine strains.

The expression system is preferably part of a plasmid which enables simple transfer of the expression system within the different vaccine strains.

The transcription module (P_{M}) comprises a regulatory sequence that controls transcription of the epitope or/and polytopic antigen or polytope of the present invention. This regulatory sequence may provide a maximum transcription rate and may be subject of downregulation in the bacterial cell, in particular in the Salmonella cell. Any suitable transcription control sequence may be used. In particular, the regulatory sequences of the Salmonella Typhimurium gene pagC can be used, which provides a moderate transcription rate which is partially suppressed under laboratory (ex vivo) conditions and becomes activated when the vaccine strain approaches a specific environment within a mammalian or avian host after oral administration of the bacterium, in particular of the Salmonella vaccine strain.

The translation module (SD) may comprise a Shine Dalgarno sequence which regulates the translation rate of the epitope or/and polytopic antigen or polytope of the present invention. Either a Shine Dalgarno sequence with high activity can be used in the expression system or it can be exchanged by a less active one in order to achieve original vitality of the vaccine strain.

The translocation module (LP) may comprise a signal peptide encoding sequence which directs the expressed polytopic antigen or polytope from the cytosol of the bacterial strain, in particular the Salmonella bacterium, into the periplasma. The signal peptide can be fused with a polypeptide (Tag) which enables immunological detection of the recombinant protein e.g. by monoclonal antibody. The signal peptide of the cholera toxin subunit B (ctxB) is preferably employed.

The antigenic module (AIV-Ag) may provide restriction sites for introducing the sequence encoding the polytopic antigen or polytope of the present invention.

The carrier module (LP:OMD) translocates the epitope or/and polytopic antigen or polytope into the outer membrane of the bacterial strain, in particular the Salmonella vaccine strain. The carrier module may comprise of a linking region (LR) which protrudes the antigen from the surface of the bacterial carrier into the surrounding environment. The carrier module may encode an outer membrane domain (OMD) capable of inserting the fusion protein into the outer membrane of the bacterial strain, in particular Salmonella vaccine strain. For example a carrier module can be introduced with an extended linking region. In another variation the linking region may contain a cleavage site for an endopeptidase. The endopeptidase may originate from the bacterial strain, in particular the Salmonella vaccine strain, e.g. the outer membrane peptidase PgtE, or from the host environment, e.g. trypsin. After peptidase cleavage, the epitope or/and polytope is released into the host environment which may improve the epitope or/and polytope-specific immune response.

The modules of the expression system are preferentially prepared by standard (bio)chemical means and inserted into a plasmid backbone via restriction sites.

The expression system can be established in bacteria, in particular gram-negative bacteria, more particular in Salmonella, even more particular in Salmonella vaccine strains. It is preferred to select the strain from the group consisting of Salmonella enterica enterica serovar Enteritidis, Salmonella enterica enterica serovar Typhimurium, Salmonella enterica enterica serovar Typhi, Salmonella enterica enterica serovar Gallinarum, Salmonella enterica enterica serovar Pullorum, Salmonella enterica enterica serovar Choleraesuis, more preferably the strain is selected from the group consisting of Salmonella enterica ssp. Enterica serovar Enteritidis: Salmovac SE, TAD vacE, serovar Typhimurium: Zoosalroal H, TAD vacT; serovar Gallinarum: Nobilis SG9R.

A preferred expression system comprises the sequence shown in SEQ ID NO:8 (FIG. 3). Another preferred expression system comprises plasmid pSS52 of FIG. 2. In this expression system a series of modules is inserted into a pKK plasmid backbone which comprises DNA sequences for plasmid replication (ORI), plasmid maintenance, e.g. the gene for α-lactamase (bla) which mediates resistance to Ampicillin, and a sequence for transcriptional termination (T).

Yet another preferred expression system comprises plasmid pFluₚₑₚ of FIG. 1.

The main part of the particular expression plasmid pFluₚₑₚ as described in FIG. 1 is the transporter unit for surface exposure of heterologous proteins in Salmonella. In pFluₚₐₚ, the transporter unit comprises a linking region (LR) and an outer membrane domain (OMD). In pFluₚₑₚ, the transporter unit originates from an autotransporter, particularly from the AIDA-I autotransporter. The AIDA-I autotransporter has proven to translocate even large antigens onto the surface of a Salmonella enterica enterica serovar Typhimurium cell.

The DNA sequence of the original autotransporter unit may be modified in order to enable modular insertion of antigens and to further optimise protein expression by the exchange of modular cassettes which control protein expression, e.g. initiation of transcription (Promoter), initiation of translation (Shine-Dalgarno sequence, SD), termination of transcription (T). In pFluₚₑₚ, the modified DNA sequence of the AIDA-I transport unit has been optimised to maintain functional integrity of the transporter unit.

The DNA sequence encoding a polytope may be inserted between the signal peptide (SP) and the linking region (LR) via two unique restriction sites (R1, R2) which generates a precursor protein comprising the signal peptide, the polytope, the linking region and the outer membrane domain (β-barrel). During secretion process the signal peptide (SP) is removed and the mature protein inserts into the outer membrane via outer membrane domain (OMD). The linking region exposes the NH₂-terminal influenza virus antigen, in particular a polytope on the bacterial surface.

The selection marker sequence may encode a protein which ensures plasmid maintenance in a bacterial host. This may be either a protein which mediates antibiotic resistance or a protein which compensates vital deficiencies of the bacterial host. ORI represents the origin of plasmid replication.

### FIGURE LEGENDS

FIG. 1 describes plasmid pFluₚₑₚ.

FIG. 2 describes plasmid pSS52.

FIG. 3 describes the Hpa-1-HindIII fragment of pSS52 (SEQ ID NO: 8).

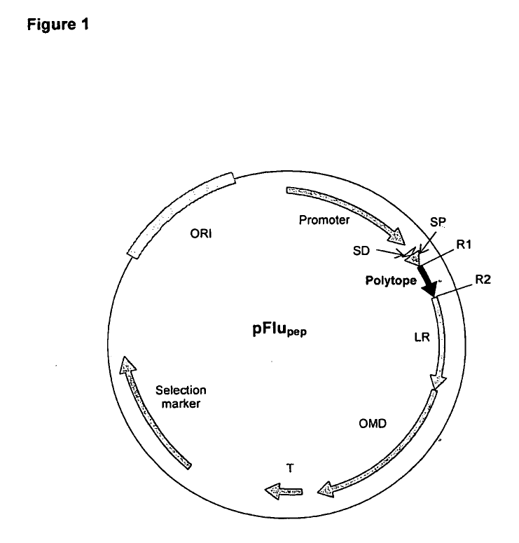

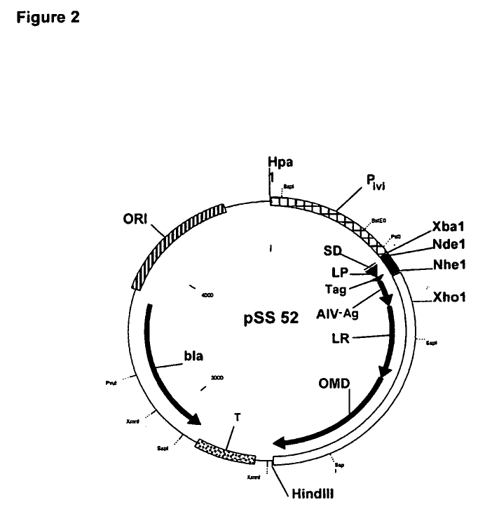

### EXAMPLES

### Example 1

Two influenza virus proteins have been exemplary selected for describing the method for identifying novel antigens for a heterosubtypic anti AI virus vaccine or/and immunogenic composition of the present invention in more detail, the hemagglutinin (HA) and the matrix protein 2 (MP2). Both proteins appear on the surface of infected cells and become accessible for the host immune system.

### 1. Identification of Potentially Essential Protein Portions of AI Virus

### Hemagglutinin (HA): Potentially Essential Protein Portion 1

Comparative analysis of HA using the amino acid sequences of various AI virus subtypes revealed a highly conserved primary' structure of hydrophobic character within a region which represents the so-called fusion peptide (FP). FP mediates the fusion between the viral membrane and the endosomal membrane during infection process. The fusion peptide is generated at the N-terminus of the HA2 subunit (HA2ₐₐ₁₋ₐₐ₂₄) by post-translational cleavage of the HA precursor protein HA0. In HA0 the cleavage site and the later fusion peptide forms a prominent surface loop in the middle of the stalk region. Comparative sequence analysis of fusion peptide from all relevant avian influenza virus subtypes yields an amino acid sequence which is prevalent in almost all AI virus subtypes.

| | | |
|---|---|---|
| [HA2_{FP1}] | G-L-F-G-A-I-A-G-F-I-E | (SEQ ID NO: 1) |

### Hemagglutinin (HA): Potentially Essential Protein Portion 2

Another region within the HA2 subunit (HA2ₐₐ₃₈₋ₐₐ₁₂₉) is also of relevance for the fusion between the viral membrane and the endosomal membrane during infection process. The indicated region contains a sub-region (HA2ₐₐ₄₀₋ₐₐ₁₀₅) which is transformed from a loop structure into a α-helical structure at low pH. The formation of this α-helix causes a conformational change within the whole HA molecule which is needed to accomplish the fusion of the viral and the endosomal membrane. The analysis of the HA2ₐₐ₄₀₋ₐₐ₁₀₅ region of all relevant AI virus subtypes by a prediction algorithm for helix structure, such as Agadir, has shown that the long α-helical stretch is made up by several clusters of 9-19 amino acids. The number, size and distribution of the individual α-helical clusters within the HA2ₐₐ₄₀₋ₐₐ₁₀₅ region were found specific for each viral H-subtype. On the basis of these data, helical clusters can be identified which are almost identical in all AI virus H-subtypes. The amino acid sequences of one of these clusters are exemplarily presented:

| | | |
|---|---|---|
| [HA2_{Helix1a}] | K-A-I-D-K-I-T-S-K | (SEQ ID NO: 2) |
| [HA2_{Helix1b}] | K-A-I-D-G-I-T-N-K | (SEQ ID NO: 3) |
| [HA2_{Helix1c}] | K-A-I-D-G-V-T-N-K | (SEQ ID NO: 4) |
| [HA2_{Helix1d}] | S-A-I-D-Q-V-T-G-K | (SEQ ID NO: 5) |

### Matrix Protein 2 (MP2): Potentially Essential Protein Portion 3

Comparative data bank analysis of MP2 revealed a highly conserved primary structure within a region representing the so-called ectodomain (eMP2). The eMP2 is relevant for membrane integration and plays a role in virion incorporation. MP2 is a trans-membrane protein and works as a (low) pH-activated ion channel that permits protons to enter the virion during uncoating and to modulate the pH of intracellular compartments which is an essential function for the prevention of acid-induced conformational changes of intra-cellularly cleaved HA0 in the trans-Golgi network.

Comparative data bank analysis using the primary structure of the eMP2 revealed 2 polypeptides which appear in the majority of presently known AI virus isolates:

| | |
|---|---|
| [eMP2₁] | |
| M-S-L-L-T-E-V-E-T-L-T-R-N-G-W-G | (SEQ ID NO: 6) |
| [eMP2₂] | |
| M-S-L-L-T-E-V-E-T-P-T-R-N-E-W-E | (SEQ ID NO: 7) |

### 2. Serial Combination of Potentially Essential Protein Portions to Form a Polytopic Antigen (Polytope)

The concept is to combine all potentially essential protein portions into one polytope which is used to immunize an avian host. Ideally, such a polytope raises an immune response which is directed against the original viral proteins. The identified protein portions are differently combined as exemplarily disclosed in the following paragraphs:

| | |
|---|---|
| Polytope 1 | [eMP2₁]:[eMP2₂]:[HA2_{FP1}] or |
| Polytope 2 | [eMP2₁]:[eMP2₁]:[eMP2₂]:[eMP2₂]:[HA2_{FP1}]:[HA2_{FP1}] or |
| Polytope 3 | [HA2_{Helix1a}]:[HA2_{Helix1b}]:[HA2_{Helix1c}]:[HA2_{Helix1d}] |

In order to avoid the generation of extra-epitopes which may result from amino acid sequences shared by proximate protein portions, a spacer sequence (SS) can be inserted between proximate protein portions. A spacer sequence may comprise the serial amino acids G-P-G-P-G.

| | |
|---|---|
| Potytope 3.1 | [HA2_{Helix1a}]:[SS]:[HA2_{Helix1b}]:[SS]:[HA2_{Helix1c}]:[SS]: |
| | [HA2_{Helix1d}] |

Additionally, an immuno-stimulatory polypeptide (IS) can be combined with a polytope in order to further improve specific immune response in an avian host. An immuno-stimulatory polypeptide may comprise the serial amino acids R-K-K-R-R-Q-R-R-R-R-A-A-A which are representing the protein transduction domain.

| | |
|---|---|
| Polytope 3.2 | [IS]:[HA2_{Helix1a}]:[SS]:[HA2_{Helix1b}]:[SS]:[HA2_{Helix1c}]:[SS]: |
| | [HA2_{Helix1d}] |

3. Identification of Polytopes which are able to Provoke Specific Immune Response Against the Original Viral Proteins

Salmonella live vaccine carriers are rendered as an ideal immune stimulant to control influenza virus replication in birds. Live-attenuated Salmonella vaccine strains of the serovars Gallinarum, Enteritidis and Typhimurium are commonly used for oral vaccination of fowls, but most of these approved vaccine strains are not tested for the expression of extra recombinant proteins. Particularly, these vaccine strains have not been tested for the surface exposure of recombinant proteins.

### Example 2

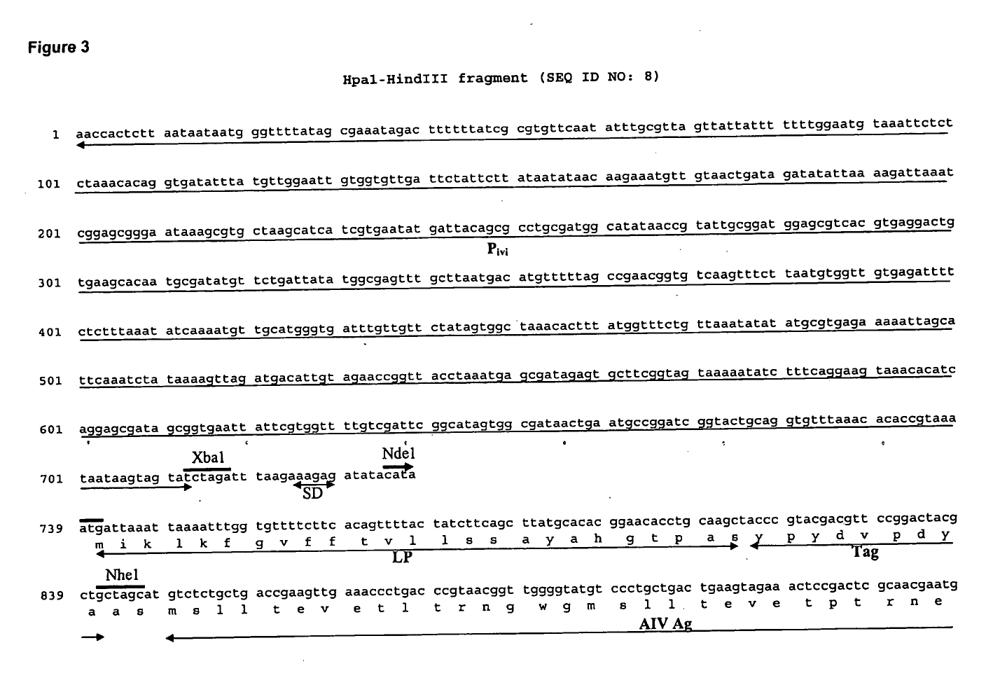

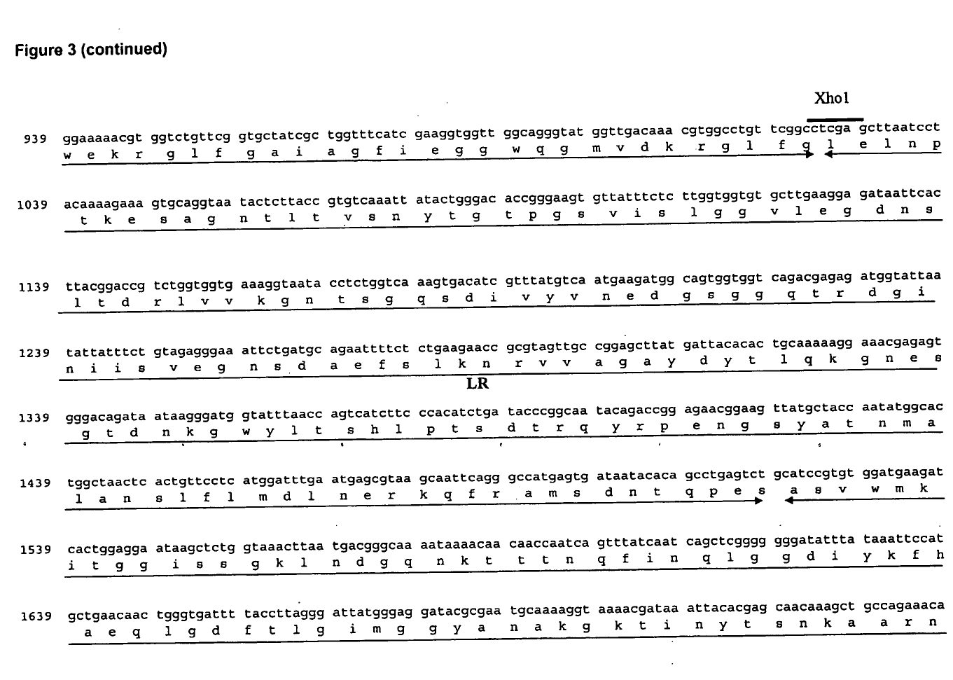

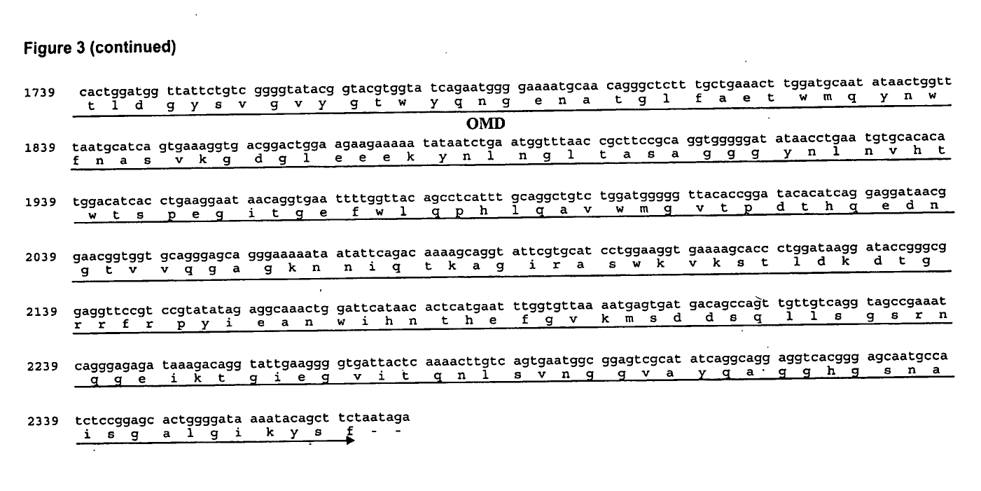

In the following paragraphs an expression system is described which is used to optimise expression of the prepared polytopes in the various Salmonella vaccine strains approved for poultry. The expression system in this example consists of different genetic modules which are used to adjust the expression and translocation of the various polytopes onto the surface of the individual Salmonella vaccine strains. The expression system of this example is part of a plasmid which enables simple transfer of the expression system within the different vaccine strains. FIG. 3 describes the expression system of this example (Hpa1-HindIII fragment of the plasmid carrying the expression system).

### Transcription Module (P_{M})

In this example, the transcription module is made up by a DNA-fragment which encodes regulatory sequences that control transcription of a succeeding gene. These regulatory sequences provide a maximum transcription rate which is reduced to an almost inactive status by specific factors produced by Salmonella cell. In the exemplified construct, an approximate 400 Bp fragment is used which contains the regulatory sequences of the Salmonella Typhimurium gene pagC. The regulatory sequences of this fragment (P_{M}) provide moderate transcription rate which is partially suppressed under laboratory (ex vivo) conditions and become activated when the vaccine strain approaches a specific environment within a mammalian or avian host after oral administration of the Salmonella vaccine strain. The transcription module is located on an Hpa1/Xba1 fragment which can be easily replaced. Thus the regulatory sequences of the Salmonella Typhimurium gene pagC can be replaced by the regulatory sequences from another gene which for example provides a higher transcription rate and/or becomes activated in a different environment of the mammalian and/or avian host.

### Translation Module (SD)

The Xba1/Nde1-fragment of the exemplified construct encodes the Shine Dalgarno sequence which regulates the translation rate of the succeeding gene. Either a Shine Dalgarno sequence with high activity can be used in the expression system of this example or it can be exchanged by a less active one in order to achieve original vitality of the vaccine strain.

### Translocation Module (LP)

In this example, the expression system comprises a signal peptide which directs the expressed protein from the cytosol of the Salmonella bacterium into the periplasma. For experimental reasons, the signal peptide can be fused with a polypeptide (Tag) which enables immunological detection of the recombinant protein by monoclonal antibody. The signal peptide of the cholera toxin subunit B (ctxB) is commonly used in the exemplified expression system. In this example, the translocation module is encoded by an Nde1/Nhe1 fragment which can be exchanged by modules of different composition.

### Antigenic Module (AIV-Ag)

In this example, the Nhe1/Xho1 sites are conceived for introducing the DNA sequence encoding the various AI virus polytopes.

### Carrier Module (LP:OMD)

In this example, the carrier module translocates the AI virus polytopes into the outer membrane of the Salmonella vaccine strains. The carrier molecule comprises of a linking region (LR) which protrudes the antigen from the surface of the bacterial carrier into the surrounding environment. The outer membrane domain (OMD) of the carrier module inserts the fusion protein into the outer membrane of the Salmonella vaccine strain. In this example, the carrier module is encoded by the Xho1-HindIII-fragment which can be exchange by carrier modules of different shape. For example a carrier module can be introduced with an extended linking region. In another variation the linking region may contain a cleavage site for an endopeptidase. The endopeptidase may originate from the Salmonella vaccine strain, e.g. the outer membrane peptidase PgtE, or from the host environment, e.g. trypsin. After cleavage, the polytope is released into the host environment which may improve polytope-specific immune response.

The modules are preferentially prepared by standard chemical means and inserted into a plasmid backbone by the indicated cloning sites. In the present example the series of modules is inserted into a pKK plasmid backbone which comprises DNA sequences for plasmid replication (ORI), plasmid maintenance, e.g. the gene for β-lactamase (bla) which mediates resistance to Ampicillin, and a sequence for transcriptional termination (T).

The expression system of the present example can be established in various Salmonella vaccine strains which differ in their infection biology and as a consequence in their competence to stimulate the host immune system.

Comparative analyses of the expression system in the various Salmonella vaccine strains under laboratory conditions confirmed surface exposure of polytope 1 in the following Salmonella vaccine strains: Salmonella enterica ssp. Enterica Enteritidis: Salmovac SE, TAD vacE, Typhimurium: Zoosalroal H, TAD vacT, Gallinarum: Nobilis SG9R.

The exposure of the AI virus polytope onto the Salmonella cell surface was demonstrated by specific binding of Tag-specific monoclonal antibody on intact cells. Moreover it was demonstrated that the surface exposed polytope can be completely removed by treating intact cells with the endopeptidase trypsin. The comparative analyses further revealed that the polytope was most efficiently expressed in the vaccine strain Salmovac SE which favours this vaccine strain for the further immunological testing of the various AI virus polytopes and as a general carrier strain for expression of recombinant proteins.

### Example 3

### Immunisation Experiments in Chicks Using Salmonella Vaccine Strains Expressing AI Virus Polytopes

In the following experimental set-ups it is sequentially tested whether a Salmonella vaccine strain expressing one of the AI virus polytopes is able to provoke in orally vaccinated chicks (1) a polytope specific immune response, (2) an immune response specific for the original AI virus protein, e.g. HA or MP2, and finally (3) an immune response which suppresses AI virus replication in a standard cell culture model or even protect vaccinated chicks against oral challenge infection with HPAI or LPAI virus. Due to the modular concept either the Salmonella vaccine strain, the expression system and the composition of the polytopes can be changed at every experimental step in order achieve optimum immune response for AI virus neutralisation.

In an exemplified immunization protocol, one day old chickens receive an oral dosis of 10⁸ colony forming units (CFU) of a given Salmonella vaccine strain carrying the outlined expression system with an AI virus polytope. Optionally, animals may receive an oral booster immunization with 10⁹ CFU of the recombinant Salmonella vaccine strain on experimental day 21 or 35. This may improve polytope specific immune response. In a second step, the serum and mucosal antibodies of the immunized animals are tested for specificity towards the original viral proteins by standard serological assays, e.g. ELISA or Immuno blotting. Moreover, it can be evaluated whether the antibodies of immunized animals are able to suppress virus replication in cell culture. In a final experimental set-up, it is tested whether vaccinated animals resist an oral challenge infection with HPAI or LPAI virus.

1. A live-attenuated bacterial cell comprising a heterologous nucleotide sequence encoding at least one influenza virus antigen in operative linkage to an expression system.

2. The bacterial cell of claim 1 which is a gram negative cell, in particular a Salmonella cell, more particular a cell selected from Salmonella enterica serovar Enteritidis, Salmonella enterica enterica serovar Typhimurium, Salmonella enterica enterica serovar Typhi, Salmonella enterica enterica serovar Gallinarum, Salmonella enterica enterica serovar Pullorum, Salmonella enterica enterica serovar Choleraesuis, Salmonella strain Nobilis SG9R, and Salmonella strain Ty21a.

3. The bacterial cell of claim 1, wherein the expression system comprises an autotransporter expression system, wherein the at least one influenza virus antigen is presented on the cell surface, in particular the expression system comprises an AIDA-I autotransporter expression system.

4. The bacterial cell of claim 3, wherein the autotransporter polypeptide expressed by the autotransporter expression system forms a fusion polypeptide with the at least one influenza virus antigen, wherein the autotransporter polypeptide is preferably located C-terminally of the at least one influenza virus antigen.

5. The bacterial cell of claim 1, wherein the expression system effects cytosolic expression of the at least one influenza virus antigen.

6. The bacterial cell of claim 1, wherein the expression system and the heterologous nucleotide sequence are located on an expression plasmid.

7. The bacterial cell of claim 1, wherein the expression system and the heterologous nucleotide sequence are stably integrated into the bacterial chromosome.

8. The bacterial cell of claim 1, wherein the at least one influenza virus antigen comprises at least one epitope of an influenza virus polypeptide.

9. The bacterial cell of claim 8, wherein the at least one epitope is selected from complete or partial sequences of influenza virus polypeptide domains which are essential for the viral function or/and which are conserved among different strains.

10. The bacterial cell of claim 8, wherein the influenza virus polypeptide is selected from hemagglutinin, neuraminidase, matrix protein M1, matrix protein M2, non-structural protein 1, non-structural protein 2, nucleoprotein, or/and polymerase complex proteins.

11. The bacterial cell of claim 8, wherein the at least one epitope independently has a length of at least 5 up to 50 amino acid residues, preferably of a at least 5 up to 20 amino acid residues, or at least 10 up to 30 amino acid residues.

12. The bacterial cell of claim 8, wherein the at least one influenza virus antigen comprises a polytope.

13. The bacterial cell of claim 12, wherein the polytope comprises 2 to 10 epitopes, 5 to 20 epitopes, or 10 to 40 epitopes.

14. The bacterial cell of claim 13, wherein at least two epitopes are selected from complete or partial sequences of variants of the same influenza virus polypeptide domain, wherein the variants are preferably obtained from different strains.

15. The bacterial cell of claim 13, wherein at least two epitopes are identical.

16. The bacterial cell of claim 1 comprising a sequence selected from SEQ ID NOs: 1-7.

17. The bacterial cell of claim 1, wherein the influenza virus is selected from the group consisting of influenza A virus subtypes, in particular selected from human influenza A virus subtypes and AI viruses, more particularly selected from LPAI or HPAI.

18. The bacterial cell of claim 1, wherein the influenza virus is selected from human influenza A virus subtypes H1N1, H1N2, H2N2, H3N2, and from avian influenza A virus subtypes H5N1, H5N2, H7N3, H7N7, H10N4 and H10N5.

19. A vector comprising a nucleotide sequence encoding at least one influenza virus antigen, in particular at least one influenza virus A antigen, in operative linkage to an autotransporter expression system.

20. The vector of claim 19 comprising a sequence selected from SEQ ID NOs: 1-7.

21. The vector of claim 19 comprising SEQ ID NO: 8.

22. The vector of claim 19 which is pFluₚₑₚ or pSS52.

23. An influenza virus antigen polytope.

24. A pharmaceutical composition comprising a bacterial cell of claim 1 together with a pharmaceutically acceptable carrier, diluent or/and adjuvant.

25. The pharmaceutical composition of claim 24 for oral administration.

26. The pharmaceutical composition of claim 24 which induces mucosal, cellular or/and humoral immunity reducing virus replication in the mucosa, e.g. in the respiratory tract or/and the gut, systemic virus replication, e.g. in the brain or/and liver, or/and virus excretion.

27. The pharmaceutical composition of claim 24 which is a vaccine, in particular a heterosubtypic vaccine.

28. The pharmaceutical composition of claim 27, which is a vaccine against avian influenza.

29. (canceled)

30. (canceled)

31. (canceled)

32. A method for the prevention, treatment or/and alleviation of an influenza, in particular influenza A, comprising administering an effective dose of a bacterial cell of claim 1 to a subject in need thereof.

33. The method of claim 32, wherein administration is oral administration.

34. The method of claim 32, wherein the vaccine induces mucosal, cellular or/and humoral immunity reducing virus replication in the mucosa, e.g. in the respiratory tract or/and the gut, systemic virus replication, e.g. in the brain or/and liver, or/and virus excretion.

35. A library of cells comprising different influenza virus antigens, in particular a library comprising cells of claim 1.

36. (canceled)

37. A screening method for the identification of an influenza virus antigen, in particular of an influenza virus A antigen, capable of inducing an immune response comprising the steps
a. providing at least two bacterial cells of claim 1 capable of expressing different candidate influenza virus antigens, in particular influenza virus A antigens,
b. administering the at least two bacterial cells of (a) to a non-human animal and
c. selecting a candidate influenza virus antigen capable of inducing an immune response.

38. The method of claim 37, wherein the at least two bacterial cells of (a) are provided in a cell library.

39. The method of claim 37, wherein the antigen is capable of inducing protective immunity against an influenza virus, preferably an influenza A virus, in particular a protective immunity against at least two influenza virus strains, preferably two influenza A virus strains.

40. The method of claim 37, wherein the animal of (b) is selected from the group consisting of mouse, rat, fowls, pig, rabbit, guinea pig.

41. The method of claim 37, wherein the administration in (b) is an oral administration.

42. The method of claim 37, wherein the immune response reduces virus replication in the mucosa, e.g. in the respiratory tract or/and the gut, systemic virus replication, e.g. in the brain or/and the liver, or/and virus excretion.

43. The method of claim 32, wherein the subject matter is a human, companion animal or/and live stock such as a pig or/and a fowl.

44. A pharmaceutical composition comprising a vector of claim 19 together with a pharmaceutically acceptable carrier, diluent or/and adjuvant.

45. A pharmaceutical composition comprising a polytope of claim 23, together with a pharmaceutically acceptable carrier, diluent or/and adjuvant.

46. A method for the prevention, treatment or/and alleviation of an influenza, in particular influenza A, comprising administering an effective dose of a vector of claim 19 to a subject in need thereof.

47. A method for the prevention, treatment or/and alleviation of an influenza, in particular influenza A, comprising administering an effective dose of a polytope of claim 23 to a subject in need thereof.


SEQUENCE LISTING (11 sequences)

SEQ ID NO: 1 - protein, 11 aa, Influenza virus
GLFGAIAGFIE

SEQ ID NO: 2 - protein, 9 aa, Influenza virus
KAIDKITSK

SEQ ID NO: 3 - protein, 9 aa, Influenza virus
KAIDGITNK

SEQ ID NO: 4 - protein, 9 aa, Influenza virus
KAIDGVTNK

SEQ ID NO: 5 - protein, 9 aa, Influenza virus
SAIDQVTGK

SEQ ID NO: 6 - protein, 16 aa, Influenza virus
MSLLTEVETLTRNGWG

SEQ ID NO: 7 - protein, 16 aa, Influenza virus
MSLLTEVETPTRNEWE

SEQ ID NO: 8 - DNA, 2378 bp, artificial
  Hpa1-HindIII fragment of plasmid pSS52
aaccactcttaataataatgggttttatagcgaaatagacttttttatcgcgtgttcaatatttgcgttagttattatttttttggaatgtaaattctctctaaacacaggtgatatttatgttggaattgtggtgttgattctattcttataatataacaagaaatgttgtaactgatagatatattaaaagattaaatcggagcgggaataaagcgtgctaagcatcatcgtgaatatgattacagcgcctgcgatggcatataaccgtattgcggatggagcgtcacgtgaggactgtgaagcacaatgcgatatgttctgattatatggcgagtttgcttaatgacatgtttttagccgaacggtgtcaagtttcttaatgtggttgtgagattttctctttaaatatcaaaatgttgcatgggtgatttgttgttctatagtggctaaacactttatggtttctgttaaatatatatgcgtgagaaaaattagcattcaaatctataaaagttagatgacattgtagaaccggttacctaaatgagcgatagagtgcttcggtagtaaaaatatctttcaggaagtaaacacatcaggagcgatagcggtgaattattcgtggttttgtcgattcggcatagtggcgataactgaatgccggatcggtactgcaggtgtttaaacacaccgtaaataataagtagtatctagatttaagaaagagatatacataatgattaaattaaaatttggtgttttcttcacagttttactatcttcagcttatgcacacggaacacctgcaagctacccgtacgacgttccggactacgctgctagcatgtctctgctgaccgaagttgaaaccctgacccgtaacggttggggtatgtccctgctgactgaagtagaaactccgactcgcaacgaatgggaaaaacgtggtctgttcggtgctatcgctggtttcatcgaaggtggttggcagggtatggttgacaaacgtggcctgttcggcctcgagcttaatcctacaaaagaaagtgcaggtaatactcttaccgtgtcaaattatactgggacaccgggaagtgttatttctcttggtggtgtgcttgaaggagataattcacttacggaccgtctggtggtgaaaggtaatacctctggtcaaagtgacatcgtttatgtcaatgaagatggcagtggtggtcagacgagagatggtattaatattatttctgtagagggaaattctgatgcagaattttctctgaagaaccgcgtagttgccggagcttatgattacacactgcaaaaaggaaacgagagtgggacagataataagggatggtatttaaccagtcatcttcccacatctgatacccggcaatacagaccggagaacggaagttatgctaccaatatggcactggctaactcactgttcctcatggatttgaatgagcgtaagcaattcagggccatgagtgataatacacagcctgagtctgcatccgtgtggatgaagatcactggaggaataagctctggtaaacttaatgacgggcaaaataaaacaacaaccaatcagtttatcaatcagctcgggggggatatttataaattccatgctgaacaactgggtgattttaccttagggattatgggaggatacgcgaatgcaaaaggtaaaacgataaattacacgagcaacaaagctgccagaaacacactggatggttattctgtcggggtatacggtacgtggtatcagaatggggaaaatgcaacagggctctttgctgaaacttggatgcaatataactggtttaatgcatcagtgaaaggtgacggactggaagaagaaaaatataatctgaatggtttaaccgcttccgcaggtgggggatataacctgaatgtgcacacatggacatcacctgaaggaataacaggtgaattttggttacagcctcatttgcaggctgtctggatgggggttacaccggatacacatcaggaggataacggaacggtggtgcagggagcagggaaaaataatattcagacaaaagcaggtattcgtgcatcctggaaggtgaaaagcaccctggataaggataccgggcggaggttccgtccgtatatagaggcaaactggattcataacactcatgaatttggtgttaaaatgagtgatgacagccagttgttgtcaggtagccgaaatcagggagagataaagacaggtattgaaggggtgattactcaaaacttgtcagtgaatggcggagtcgcatatcaggcaggaggtcacgggagcaatgccatctccggagcactggggataaaatacagcttctaataga

SEQ ID NO: 9 - protein, 544 aa, artificial
  Synthetic Construct
MIKLKFGVFFTVLLSSAYAHGTPASYPYDVPDYAASMSLLTEVETLTRNGWGMSLLTEVETPTRNEWEKRGLFGAIAGFIEGGWQGMVDKRGLFGLELNPTKESAGNTLTVSNYTGTPGSVISLGGVLEGDNSLTDRLVVKGNTSGQSDIVYVNEDGSGGQTRDGINIISVEGNSDAEFSLKNRVVAGAYDYTLQKGNESGTDNKGWYLTSHLPTSDTRQYRPENGSYATNMALANSLFLMDLNERKQFRAMSDNTQPESASVWMKITGGISSGKLNDGQNKTTTNQFINQLGGDIYKFHAEQLGDFTLGIMGGYANAKGKTINYTSNKAARNTLDGYSVGVYGTWYQNGENATGLFAETWMQYNWFNASVKGDGLEEEKYNLNGLTASAGGGYNLNVHTWTSPEGITGEFWLQPHLQAVWMGVTPDTHQEDNGTVVQGAGKNNIQTKAGIRASWKVKSTLDKDTGRRFRPYIEANWIHNTHEFGVKMSDDSQLLSGSRNQGEIKTGIEGVITQNLSVNGGVAYQAGGHGSNAISGALGIKYSF

SEQ ID NO: 10 - protein, 5 aa, artificial
  Spacer
GPGPG

SEQ ID NO: 11 - protein, 13 aa, artificial
  protein transduction domain
RKKRRQRRRRAAA